(19) United States
(12) Patent Application Publication
(10) Pub. No.: US 2009/0098228 A1
(43) Pub. Date: Apr. 16, 2009

(54) AGENT AND METHOD FOR IMPROVEMENT OF IMPAIRMENT OF LEARNING AND MEMORY

(75) Inventors: Yasushi IKARASHI, Inashiki-gun (JP); Masahiro Tabuchi, Inashiki-gun (JP)

Correspondence Address: OBLON, SPIVAK, MCCLELLAND MAIER & NEUSTADT, P.C., 1940 DUKE STREET, ALEXANDRIA, VA, 22314, US

(73) Assignee: TSUMURA & CO., Tokyo (JP)

(21) Appl. No.: 11/870,079

(22) Filed: Oct. 10, 2007

(51) Int. Cl. A61K 36/232; A61K 36/74; A61P 43/00

(52) U.S. Cl. 424/773

(57) ABSTRACT

An improving agent for impairment of learning and memory containing yokukansan is provided as an easy and simple means for enhancing memory of aged people, etc. and for improving the impairment of learning and memory.

### BACKGROUND OF THE INVENTION

1. Field of the Invention

The present invention relates to an agent for improvement of impairment of learning and memory and also to a method for improvement of impairment of learning and memory. In particular, it relates to an agent and a method to suppress a decline in learning and memory which progresses with aging.

2. Description of the Related Art

It has been widely known that, as a result of aging, memorizing becomes difficult gradually even when one sees/hears the same thing repeatedly. Such a decline in learning and memory has been widely generated regardless of dementia.

It may be human nature to try to learn new knowledge not only in adolescence but also in middle age and older. Actually however, it is often difficult to learn new knowledge in middle age and older due to the decline in the learning and memory.

Consequently, a means for improving memory of people in middle age and older has been desired. However, there has been few known means for improvement of impairment of learning ability and memory under present circumstances.

### SUMMARY OF THE INVENTION

Accordingly, an object of the present invention is to provide a means for enhancing the memory of aged and other people and for improving the impairment of learning and memory with a simple way.

In order to accomplish the above and other objects, the present inventors have investigated an improving effect on impairment of learning and memory of various pharmaceuticals through the use of test methods for evaluation of an improving effect on impairment of learning and memory in experimental animals. As a result, they found an excellent improving effect on impairment of learning and memory in yokukansan which has been known as a traditional Japanese medicine (Kampo medicine in Japanese). Based on the finding, the present invention has been achieved.

Thus, the present invention provides an improving agent for impairment of learning and memory comprising yokukansan.

The present invention is also directed to a method for improving the impairment of learning and memory by administering yokukansan to a person with a decline in learning and memory.

In accordance with the present invention, it is possible to suppress a decline in learning and memory in middle aged and older people. Therefore, it is possible to provide a pharmaceutical and a method which are effective for the treatment of impairment of learning and memory of such people.

The pharmaceutical and the treatment method of the present invention have an excellent safety profile with few problem such as side effect, since the active ingredient is yokukansan which has been used traditionally as a prescription in Japan and China.

### BRIEF DESCRIPTION OF THE DRAWINGS

FIG. 1 shows the typical behavioral traces (swimming) of mice in each group on Days 1 and 5 of the training. From the upper, traces of healthy mice, amyloid β (+) mice, and amyloid β (+) mice administered with TJ-54 are shown.

FIG. 2 shows the latency time of mice in each group on Day 5 of the training. Each data is shown as the mean±SE. Significance among the groups was evaluated by a Mann-Whitney's U test. *p<0.05 vs Aβ (−), ⁵⁵⁴ p<0.05 vs Aβ (+).

FIGS. 3(A) and 3(B) show the results of retention trials in a step-through passive-avoidance test. FIG. 3(A) shows the result on Day 15 and FIG. 3(B) shows the result on Day 29. Each data is shown as the mean±SE. Significance among the groups was evaluated by Fisher's PLSD multiple comparison test after a one-way analysis of variance (one-way ANOVA). ***p <0.001 vs control+DW; ^{†}*p<0.05 and ^{†\}p<0.01 vs TD+DW. Numbers in the parentheses show the animal numbers which entered a dark chamber in each group.

### DETAILED DESCRIPTION OF THE PREFERRED EMBODIMENTS

Yokukansan used in the present invention contains Atractylodes lancea rhizome, Poria sclerotium, Uncaria hook, Cnidium rhizome, Japanese Angelica root, Bupleurum root, Glycyrrhiza, etc. as crude drugs, but not limited thereto. Yokukansan is utilized in a treatment of insomnia, neurosis including anxiety, and night crying and irritability of children.

In addition, it has been reported that yokukansan shows an improving effect on excitation and aggression occurred as peripheral symptoms of dementia including Alzheimer's disease (Iwasaki, et al., J. Clin. Psychiatry, 66:248-252, 2005). With regard to the anti-anxiety action of yokukansan, a study using mice has been reported (Kuribara, et al., Jpn. J. Neuropsychopharmacol., 18:179-190, 1996).

The compounding range (composition) of crude drugs in yokukansan is generally as shown in Table 1. Incidentally, in yokukansan, additional formulations such as yokukansan-added chimpi (dried peel of mandarin orange) and hange (rhizome of Pinellia ternate) are also available and they are also able to be used as the improving agent for impairment of learning and memory according to the present invention.

TABLE 1

| Names | Compounding |
|---|---|
| Atractylodes lancea rhizome (JP) | 4.0 g |
| Poria sclerotium (JP) | 4.0 g |
| Cnidium rhizome (JP) | 3.0 g |
| Japanese Angelica root (JP) | 3.0 g |
| Bupleurum root (JP) | 2.0 g |
| Glycyrrhiza (JP) | 1.5 g |
| Uncaria hook (JP) | 3.0 g |

In the improving agent for impairment of learning and memory of the present invention, either of yokukansan having the above composition itself and an extract can be used as an active ingredient and made into a pharmaceutical preparation using known carriers for pharmaceuticals.

An extract of yokukansan can be prepared according to classical methods in which a mixture of the crude drugs of the compounding ratio as mentioned above is extracted with an appropriate aqueous solvent in 5- to 20-fold amount followed by subjecting to a solid-liquid separation. Examples of the preferred aqueous solvent are water, ethanol and aqueous solution of acetic acid. The method of the extraction can be any of hot extraction and cold extraction. In particular, a method wherein water is used as a solvent and extraction is conducted at 90 to 100° C. is preferred.

The yokukansan extract thus obtained may be dried to be made into powder if necessary. Although the extract or the powder may be directly administered as it is, it may be also able to be made into an extract preparation in view of easy administration and convenient carrying. For example, a mixture of the crude drugs is extracted with 10-fold amount of hot water followed by subjecting to a solid-liquid separation. The obtained extract liquid is concentrated and dried, and the resulting dried extract of yokukansan is made into a preparation such as diluted powder, granules, tablets and capsules according to conventional methods by being mixing with appropriate fillers (such as lactose, corn starch and crystalline cellulose), excipients, etc., which have been commonly used for pharmaceutical preparations.

The improving agent for impairment of learning and memory according to the present invention may be preferably used as an oral preparation.

Examples of the oral preparation include solid ones such as in the form of powder, granules, tablets and capsules and liquid ones such as suspension, emulsion, syrup and elixir, wherein pharmaceutical carriers corresponding to the administration form and dosage form thereof may be used.

For example, in the case of solid oral preparations, there may be used starch, lactose, sugar, mannitol, carboxymethyl cellulose, corn starch, inorganic salt, etc. Further, optional components such as binder, disintegrating agent, surfactant, lubricant, fluidity accelerator, corrigent, coloring agent and perfume may be compounded therewith as needed.

Among the above optional components, examples of the binder include starch, dextrin, acacia, gelatin, hydroxypropyl starch, methyl cellulose, carboxymethyl cellulose sodium, hydroxypropyl cellulose, crystalline cellulose, ethyl cellulose, polyvinylpyrrolidone and Macrogol.

Examples of the disintegrating agent include starch, hydroxypropyl starch, carboxymethyl cellulose sodium, carboxymethyl cellulose calcium, carboxymethyl cellulose and low-substituted hydroxypropyl cellulose.

Examples of the surfactant include sodium laurylsulfate, soybean lecithin, sucrose fatty acid ester and Polysorbate 80. Examples of the lubricant include talc, waxes, hydrogenated vegetable oil, sucrose fatty acid ester, magnesium stearate, calcium stearate, aluminum stearate and polyethylene glycol. Examples of the fluidity accelerator include light silicic acid anhydride, dried aluminum hydroxide gel, synthetic aluminum silicate and magnesium silicate.

A dose of the improving agent for impairment of learning and memory in accordance with the present invention to persons with a decline in learning and memory due to aging or Alzheimer's disease varies depending upon the administering route, the age of the subject, the degree of impairment, etc. However, in the case of oral administration, it is usually 2.5 g to 25.0 g of a yokukansan preparation (TJ-54; manufactured by Tsumura & Co.) corresponding to 1.0 g to 10.0 g of a dried extract of yokukansan or, preferably, 7.5 g (3.25 g of a dried extract of yokukansan ) for an adult per day by dividing into two or three times before or between meals.

### EXAMPLES

The present invention is illustrated in more detail by way of the following Examples which are not intended to be limiting of the present invention.

### Example 1

### Test for Improvement of Impairment of Learning Ability

Female mice in which human β-amyloid is expressed (β-amyloid precursor protein gene transgenic mice; Aβ(+)APP-Tg mice) were purchased from Taconic Farms Inc., and used as experimental animals, and Aβ(−)APP-Tg mice in which Aβ is not expressed were used as a control. These animals were divided into the four groups as follows. Numbers in the parentheses show the numbers of the animals used.

Group 1: Aβ(−)mice fed with normal feed (n=8)

Group 2: Aβ(+)mice fed with normal feed (n=12)

Group 3: Aβ(+)mice fed with a feed mixed with 0.5% TJ-54 (n=12)

Group 4: Aβ(+)mice fed with a feed mixed with 1.0% TJ-54 (n=9)

The Aβ(−)mice of Group 1 and the Aβ(+)mice of Group 2 were fed with a normal feed (MF; manufactured by Oriental Yeast Co., Ltd.) and the Aβ(+)mice of Groups 3 and 4 were fed with a mixed feed wherein 0.5% or 1.0% of yakukansan extract (TJ-54; manufactured by Tsumura & Co.) was mixed with the normal feed for six months (from 5 to 11 month-old). During the experimental period, 2 to 4 animals per cage were bred under the feeding environment at a temperature of 23÷2° C., a relative humidity of 55±5% and illumination for 12 hours (light period: 7:00 to 19:00), in a plastic cage for mice (type M-55TG; 230×310×155 mm; Okazaki Sangyo). A daily dose of TJ-54 calculated from the daily ingesting amount of the feed was 1.3 g/kg for a group of 0.5% TJ-54 and was 2.5 g/kg for a group of 1.0% TJ-54 group.

Then a test for acquisition of memory (learning ability) was conducted using a Morris-type water maze apparatus. Specifically, one platform (the platform is hidden at such a water level that mice are able to have a rest thereon) was installed in a basin-like water tank. When a training where mice were subjected to swimming in such an apparatus (once daily; for one minute) was conducted for five days, the mice recognized the position of the platform and the time required for arriving there became gradually shorter. The time until the mice arrived the platform (latency time) was measured and used as an index for acquisition of memory.

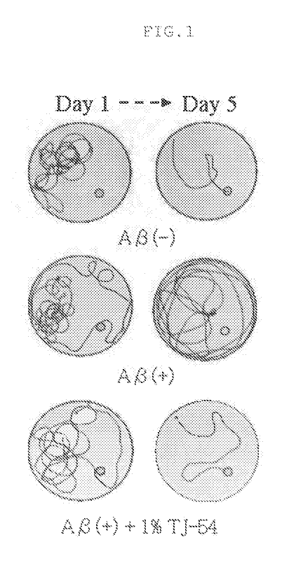

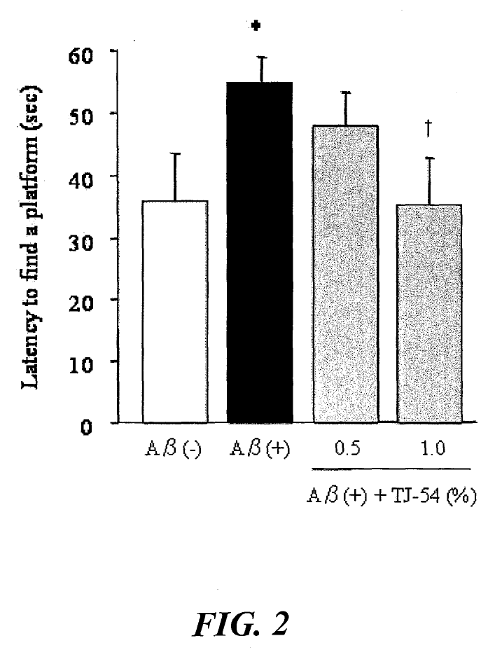

Loci of typical behavior (swimming) of the mice in each group on Days 1 and 5 of the training are shown in FIG. 1. On Day 1, any of the groups hardly arrived the platform. However, as shown in FIG. 2, when the trainings were continuously conducted for five days, the Aβ(−)mice (control) learned the position of the platform and arrived there within about 35 seconds, whereas the Aβ(+)mice were slow to learn the position despite the repeated trainings and continued to swim during nearly 60 seconds. On the other hand, when TJ-54 was administered, the latency time of the Aβ(+)mice shortened in a dose-dependent manner. From those results, it was found that yokukansan (TJ-54) had an improving effect on impairment of learning ability.

### Example 2

### Test for Improvement of Impairment of Memory

A step-through passive-avoidance test was conducted using male Wistar rats (three-week-old; body weight: 35 to 45 g) to evaluate the functions of memory acquirement and retention. The rats purchased from Charles Liver laboratories Japan, Inc. were bred for one week for habituation and those of four-week-old (body weight: 79 to 98 g) were used for the experiment. Throughout the habituation and experimental period, the animals were separately bred in a stainless steel cage (type RBC-12; 260×380×200 mm; Ishihara Co., Ltd.) under a breeding environment at a temperature of 23±2° C., a relative humidity of 55±5% and illumination for 12 hours (light period: 7:00 to 19:00).

Firstly, all of those experimental animals were subjected to an acquisition trial for step-through passive-avoidance memory under a breeding condition with a normal feed (MF) for five days (once daily).

An apparatus (AP model; O'Hara Co., Ltd.) for step-through passive-avoidance test consisting of two compartments, that is, a bright chamber (200 mm length×100 mm width×190 mm height; illuminance: 27 watts, 3,000 luxes) and a dark chamber (200 mm×230 mm'190 mm) was used. These compartments were connected by a guillotine door. The dark chamber had a grid floor and arbitrary turning-on of electricity was possible.

The acquisition trial for memory was carried out as follows. When a rat was placed in the bright chamber where a guillotine door was in an open state, the rat soon passed through the opened door and entered the dark chamber. Three seconds after the animal entirely entered the dark chamber, electric foot shock (0.3 mA for 3 seconds) was delivered to the floor grid in the dark chamber. Then, the animal returned to the bright chamber to avoid the electric shock. The time spent in the bright chamber was measured as a latency time. Such an acquisition trial was conducted once daily and for consecutive five days whereupon the rat placed in the bright chamber no longer came into the dark chamber. When the latency time exceeded 300 seconds, the rat was judged as having learned the avoidance memory from the foot shock.

As a result, in all rats divided into four groups, the latency time exceeded 300 seconds by repeated acquisition trials for five times under a breeding with a normal feed. In the acquisition trial for five days, there was no significant difference among the groups in the latency time by elapsed days or avoidance learning process. These results showed that learning ability of the rats was identical among the groups.

After the rats in each group acquired the memory in the manner described above, Group 1 was switched to a control feed while Groups 2 to 4 were switched to a thiamine-deficient feed (TD) and breeding was conducted for 37 days (the date of switching the feed was Day 0 and the breeding was done until Day 37). Further, after switching the feed, during 37 days from Day 0 to Day 37, 10 mL of distilled water per kg of body weight (10 mL/kg) was given to Groups 1 and 2 while 0.5 g and 1.0 g of TJ-54 suspended in 10 mL of distilled water (0.5 and 1.0 g/kg) was given to Groups 3 and 4 respectively, orally once daily.

Group 1: control feed and distilled water (n=10)

Group 2: TD feed and distilled water (n=11)

Group 3: TD feed and 0.5 g/kg TJ-54 (n=10)

Group 4: TD feed and 1.0 g/kg TJ-54 (n=11)

The rats of Group 1 were fed with the control feed which corresponded to the feeding amount for Group 2. Accordingly, Group 1 is a pair-fed control group of the Group fed with TD feed and distilled water (Group 2).

The normal feed (MF), TD feed (specially ordered feed) and AIN-93G (control feed) were purchased from Oriental Yeast Co., Ltd. The TD feed was a product in which thiamine (vitamin B₁) was removed during the manufacturing process of AIN-93G. An amount of vitamin B₁ in each of the normal feed and the control feed was 0.5 mg per 100 g of the feed. No thiamine was detected from the TD feed in the measurement after manufacturing.

Retention trial for confirming the memory-retaining ability was conducted as follows. The rat which acquired the avoidance memory as above was placed in a bright chamber of the same avoidance apparatus on Days 15 and 29 from the final acquisition trial and the latency times were measured. When the latency time exceeded 300 seconds in the retention trial, the rat was judged as retaining the avoidance memory.

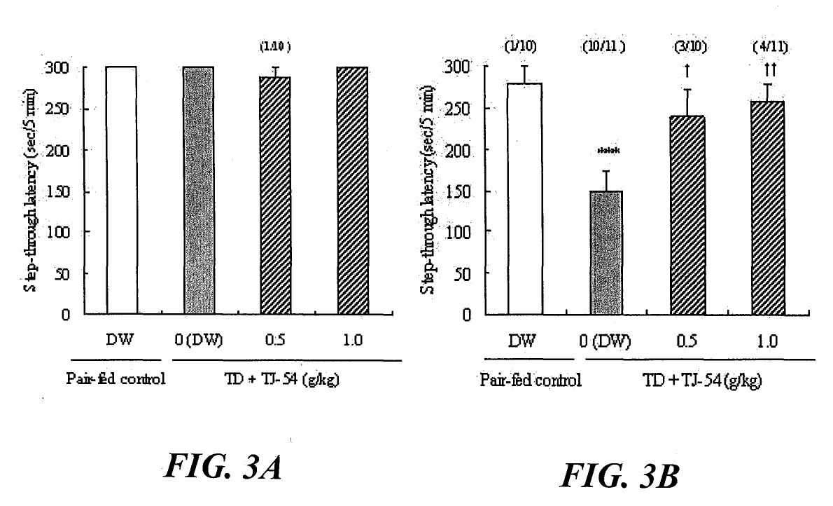

The results of the retention trials (confirmation test for retained state of memory) on Day 15 and Day 29 are shown in FIGS. 3(A) and 3(B), respectively.

On Day 15, as shown in FIG. 3(A), Group 3 (the group fed with TD feed and 0.5 g/kg TJ-54) showed a slight decrease in latency time since an animal came into the dark chamber but, in the other groups, all animals retained the latency time exceeding 300 seconds and there was no significant difference in the latency time among the groups. As a result, it was determined that, in all of the groups, memory was retained until Day 15.

On Day 29, as shown in FIG. 3(B), 1/10 of the animals entered the dark chamber in Group 1 (the group fed with the control feed and distilled water) while, in Group 2 (the group fed with TD feed and distilled water), 10/11 of the animals entered the dark chamber and the latency time was significantly shortened. In Group 3 (the group fed with TD feed and 0.5 g/kg TJ-54) and Group 4 (the group fed with TD feed and 1.0 g/kg TJ-54), the numbers of the animals coming into the dark chamber were 3 to 4 and the latency time of those TJ-54-fed groups was significantly longer than that of the TD-fed group (Group 2). As a result, it was found that, in the groups fed with yokukansan (TJ-54), the dose of 0.5 g/kg or more suppressed impairment of retention of memory (amnesia) of the TD rats.

From those results, yokukansan was found to have an improving effect on impairment of memory caused by deficiency of thiamine.

As mentioned above, the agent for impairment of learning and memory of the present invention improves the impairment of learning and memory. Further, since Yokukansan which has been used traditionally is comprised in as an active ingredient, the agent is able to be advantageously used for maintaining learning and memory in middle aged and older people suffering from a decline in the ability, and also for improvement of impairment of learning and memory caused by cerebral diseases and the like.

What is claimed is:

1. An improving agent for impairment of learning and memory comprising yokukansan.

2. The improving agent for impairment of learning and memory according to claim 1, by which yokukansan is administered in a dose of 1.0 g to 10.0 g per day in terms of the amount of extract thereof.

3. A method for improvement of impairment of learning and memory, comprising administering yokukansan to a person with a decline in learning and memory.

4. The method for improvement of impairment of learning and memory according to claim 3, wherein the decline in learning and memory is caused by aging or by deficiency of thiamine.

5. The method for improvement of impairment of learning and memory according to claim 3, wherein the decline in learning and memory is caused by Alzheimer's disease.

6. The method for improvement of impairment of learning and memory according to claim 3, wherein yokukansan is administered in a dose of 1.0 g to 10.0 g per day in terms of the amount of an extract thereof.

7. The method for improvement of impairment of learning and memory according to claim 4, wherein yokukansan is administered in a dose of 1.0 g to 10.0 g per day in terms of the amount of an extract thereof.

8. The method for improvement of impairment of learning and memory according to claim 5, wherein yokukansan is administered in a dose of 1.0 g to 10.0 g per day in terms of the amount of an extract thereof.
